(19) United States
(12) Patent Application Publication
(10) Pub. No.: US 2024/0238386 A1
(43) Pub. Date: Jul. 18, 2024

(54) METHODS FOR INCREASING RED BLOOD CELL HALF-LIFE AND ASSOCIATED TREATMENT OF ANEMIA

(71) Applicant: Marshall University Research Corporation, Huntington, WV (US)

(72) Inventors: Joseph I. Shapiro, Huntington, WV (US); Jiang Liu, Huntington, WV (US)

(21) Appl. No.: 18/560,230

(22) PCT Filed: May 10, 2022
(86) PCT No.: PCT/US22/28518

§ 371 Date: Nov. 10, 2023

Related U.S. Application Data
(60) Provisional application No. 63/187,181, filed on May 11, 2021.
(60) Provisional application No. 63/331,523, filed on Apr. 15, 2022.

(51) Int. Cl. A61K 38/46; A61P 7/06

(52) U.S. Cl. CPC A61K 38/46; A61P 7/06

(57) ABSTRACT

Methods for treating anemia are provided and include the steps of identifying a subject as having anemia characterized by a reduced number of red blood cells and then administering a polypeptide antagonist of a Na/K ATPase/Src receptor complex to the subject. Methods for increasing red blood cell half-life are also provided in which the polypeptide antagonist of a Na/K ATPase/Src receptor complex is either administered to a subject in need thereof or is made to contact a red blood cell.

### RELATED APPLICATIONS

This application claims priority from U.S. Provisional Application Ser. No. 63/187,181, filed May 11, 2021, and from U.S. Provisional Application Ser. No. 63/331,523, filed Apr. 15, 2022, the entire disclosures of which are incorporated herein by this reference.

### GOVERNMENT INTEREST

This invention was made with government support under grant numbers R15 1R15DK106666, 1R15HL150721, HL071556, and 1P20GM121299-01 awarded by the National Institutes of Health. The government has certain rights in the invention.

### TECHNICAL FIELD

The presently-disclosed subject matter generally relates to compositions and methods for treating anemia and increasing red blood-cell half-life. In particular, certain embodiments of the presently-disclosed subject matter relate to polypeptides and methods for using the polypeptides to treat anemia and to increase red blood cell half-life.

### BACKGROUND

Anemia is a complication in patients with chronic kidney disease (CKD) that is associated with poor outcomes, increased cardiovascular disease, and mortality. The mechanisms of anemia are multifactorial, including, but not limited to, erythropoietin deficiency, iron deficiency, and red blood cell (RBC) lifespan, as well as inflammatory cytokines and reactive oxygen species (ROS). Na/K-ATPase is important for many physiological processes such as stabilizing resting membrane potential and cell volume. Other than functioning as the primary ion transporter, the Na/K-ATPase also functions as a signal transducer to execute signaling functions through protein-protein interactions with other signaling molecules such as c-Src and PI3K, which has been evolved as a therapeutic target. Other than its specific ligand cardiotonic steroids (CTS), which can stimulate ROS generation through activation of the Na/K-ATPase/c-Src signaling, an increase in ROS alone can also activate the Na/K-ATPase/c-Src signaling to generate more ROS. This CTS- and ROS-mediated Na/K-ATPase/c-Src signaling-ROS axis forms a feed-forward oxidant amplification loop for Na/K-ATPase signaling and ROS production. To that end, a peptide named pNaKtide (SEQ ID NO: 5) has been developed from the N domain of the Na/K-ATPase al subunit and which can bind to the c-Src kinase domain to prevent c-Src phosphorylation and ultimately inhibit the oxidant amplification loop. Inhibition of this oxidant amplification loop effectively ameliorates ⅚^{th} partial nephrectomy (PNx)-induced uremic cardiomyopathy, anemia, and inflammatory cytokine production. Interestingly, in those studies, while PNx-induced anemia was restored with pNaKtide, induction of hemeoxygenase-1 (HO-1) did not ameliorate PNx-induced anemia.

Accordingly, further methods for treating anemia, including anemia associated with chronic kidney disease and the decreased red blood cell population observed with such anemia, would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes compositions and methods for treating anemia and increasing red blood-cell half-life. In some embodiments, a method for treating anemia is provided that includes the steps of identifying a subject as having anemia that is characterized by a reduced number of red blood cells and administering a polypeptide antagonist of a Na/K ATPase/Src receptor complex to the subject. In some embodiments, the polypeptide anatagonist comprises the sequence of SEQ ID NO: 1, or a functional fragment, and/or functional variant thereof. In some embodiments, the polypeptide comprises the sequence of SEQ ID NO: 5. In some embodiments, the polypeptide antagonist further includes a cell penetrating peptide, such as, in certain embodiments, a cell penetrating polypeptide having an amino acid sequence selected from the group consisting of SEQ ID NOS: 2-4.

With regard to the administration of the polypeptide antagonist, in some embodiments, the administering step includes oral administration, transdermal administration, administration by inhalation, nasal administration, topical administration, intraaural administration, rectal administration, intravenous administration, intramuscular administration, subcutaneous administration, intravitreous administration, subconjunctival administration, intracameral administration, intraocular administration or combinations thereof. In some embodiments, the polypeptide antagonist is administered in an amount sufficient to increase a hematocrit level in the subject, in an amount sufficient to reduce an amount of reticulocytes in the subject, in an amount sufficient to increase a half-life of a red blood cell in the subject, and/or in an amount sufficient to decrease an amount of eryptosis in the subject.

In some embodiments, the subjects to be treated in accordance with the presently-disclosed subject matter are human subjects. In some embodiments, the subject has kidney disease. In some embodiments, the anemia is chronic kidney disease-induced anemia.

Further provided, in some embodiments of the presently-disclosed subject matter, are methods for increasing red blood cell half-life. In some embodiments, a method for increasing red blood cell half-life is provided that comprises administering a polypeptide antagonist of a Na/K ATPase/Src receptor complex to a subject in need thereof, where the polypeptide anatagonist has the sequence of SEQ ID NO: 1, or a functional fragment, and/or functional variant thereof, and where the polypeptide antagonist further includes a cell penetrating peptide. In some embodiments, the polypeptide anatagonist comprises the sequence of SEQ ID NO: 5. In some embodiments, the cell penetrating polypeptide has an amino acid sequence selected from the group consisting of SEQ ID NOS: 2-4.

Still further provided, in some embodiments of the presently-disclosed subject matter are methods for increasing red blood cell half-life that include contacting a red blood cell with a polypeptide antagonist of a Na/K ATPase/Src receptor complex, where the polypeptide anatagonist has the sequence of SEQ ID NO: 1, or a functional fragment, and/or functional variant thereof, and where the polypeptide antagonist further includes a cell penetrating peptide. Such polypeptide antagonists are again inclusive of polypeptides having the sequence of SEQ ID NO: 5 and, in some embodiments, the cell penetrating polypeptide has an amino acid sequence selected from the group consisting of SEQ ID NOS: 2-4.

Further features and advantages of the present invention will become evident to those of ordinary skill in the art after a study of the description, figures, and non-limiting examples in this document.

### BRIEF DESCRIPTION OF THE DRAWINGS

In the accompanying drawings, the following group numbering and naming is used for all figures: (1) Sham (S); (2) Sham+pNaKtide (S+P); (3) ⅚th PNx (PNx); (4) PNx+pNaKtide (PNx+P); (5) PNx+Naktide; and (6) PNx+scramble pNaKtide (sc-pNaKtide).

FIGS. 1A-1B are graphs showing ⅚th partial nephrectomy (PNx)-induced decrease in hematocrit (Hct) and increase in reticulocyte count were corrected by administration of pNaKtide. In FIG. 1A, blood samples were collected on Day 0 (before Sham and PNx surgery) and Day 14 and 28 (post-second surgery). Hct (%) levels were significantly decreased on Day 14 and 28 in PNx group compared with the Sham group, which was corrected by pNaKtide administration. For Days 0 and 14 of FIG. 1A, S=12, S+P=7, PNx=12, PNx+P=13, PNx+NaKtide=5, and PNx+scramble pNaKtide=6. For Day 28 of FIG. 1A, S=20, S+P=12, PNx=24, PNx+P=20, PNx+NaKtide=5, and Nx+scramble pNaKtide=6. In FIG. 1B, blood samples were collected on Day 0 and Day 28 and stained with Thiazole Orange for reticulocyte count by flow cytometry. Representative flow cytometry histographies with negative control are shown in FIG. 1B alone with a statistical analysis graph (S=12, S+P=7, PNx=12, PNx+P=13, PNx+NaKtide=5, and PNx+scramble pNaKtide=6. *, p<0.05 vs. Sham; **, p<0.01 vs. Sham; #, p<0.01 vs. PNx, ##, p<0.01 vs. PNx).

FIGS. 2A-2C are graphs showing PNx-induced decrease in RBC half-life was corrected by administration of pNaKtide, where EZ-Link Sulfo-NHS Biotin (1.0 mg biotin/25 g body weight) was administrated via cardiothoracic puncture 3-days post second surgery and blood samples were collected from the submandibular vein on Days 3, 7, 14, 21, and 28 post-biotin injection, and where biotinylated RBCs were further labelled with Streptavidin-PE and measured with flow cytometry. FIG. 2A shows representative flow cytometry gating and histogram with negative control. FIG. 2B shows statistical analysis of biotin-positive RBCs vs. total RBCs (%) on Days 3, 7, 14, 21, and 28 post-biotin injection. For Day 3 in FIG. 2B, S=16, S+P=12, PNx=19, PNx+P=20, PNx+NaKtide=5, and PNx+scramble pNaKtide=6. For Day 7 in FIG. 2B, S=11, S+P=12, PNx=15, PNx+P=13, PNx+NaKtide=5, and PNx+scramble pNaKtide=6. For Day 14 in FIG. 2B, S=19, S+P=12, PNx=17, and PNx+P=20, PNx+NaKtide=5, and PNx+scramble pNaKtide=6. For Day 21 in FIG. 2B, S=20, S+P=12, PNx=17, and PNx+P=20, PNx+NaKtide=5, and PNx+scramble pNaKtide=6. For Day 28 in FIG. 2B, S=20, S+P=12, PNx=17, and PNx+P=20, PNx+NaKtide=5, and PNx+scramble pNaKtide=6. FIG. 2C shows a linear regression measurement of RBC half-life (T½), which was calculated by performing a least squares linear regression on the log 2 of the fraction of biotinylated RBCs vs. time **, p<0.01 vs. Sham; ##, p<0.01 vs. PNx.

FIGS. 3A-3B are graphs and images showing PNx and pNaKtide administration did not significantly change iron homeostasis but increased plasma EPO level. In FIG. 3A, the plasma iron level, TIBC, UIBC, and iron saturation rate were measured, and no significant difference was observed amongst groups (S=13, S+P=10, PNx=20, PNx+P=17, PNx+NaKtide=4, and PNx+scramble pNaKtide=6). In FIG. 3B, the left panel further shows a representative immunoblot (upper) and PVDF membrane reversible protein stain or Novex Reversible Membrane Protein Stain as loading control (lower). The right panel shows statistical analysis (S=10, S+P=6, PNx=10, and PNx+P=10, PNx+NaKtide=4, and PNx+scramble pNaKtide=5. *, p<0.01 vs. Sham; **, p<0.01 vs. Sham; #, p<0.01 vs. PNx).

FIGS. 4A-4B are graphs showing PNx-stimulated eryptosis corrected by pNaKtide administration, where blood samples were collected on Days 0 and 28, and where samples were reacted with Annexin V-APC and Annexin V binding were assessed by flow cytometry. FIG. 4A shows representative flow cytometry scatterplots with negative control, where quadrant 1 shows Annexin V binding-positive population, quadrant 2 shows Annexin V binding-negative and 7-AAD-negative population, and quadrants 3 and 4 show 7-AAD-positive populations. FIG. 4B shows the accompanying statistical analysis, where for both Days 0 and 28, S=12, S+P=7, PNx=12, and PNx+P=13, PNx+NaKtide=5, and PNx+scramble pNaKtide=6 (**, p<0.01; ##, p<0.01 vs. PNx).

FIG. 5 includes an image and a graph showing PNx-induced protein carbonylation of plasma samples corrected by administration of pNaKtide, where protein carbonylation of plasma samples was measured, where a representative immunoblot (upper) and PVDF membrane reversible protein stain or Novex Reversible Membrane Protein Stain as loading control (lower) is shown along with a statistical analysis figure (S=9, S+P=8, PNx=11, and PNx+P=10, PNx+NaKtide=5, and PNx+scramble pNaKtide=6. **, p<0.01 vs. Sham; #, p<0.01 vs. PNx).

FIG. 6 is a graph showing PNx-induced impairment of renal function and the effects of administration of peptides, where plasma BUN was measured with a commercially-available assay kit according to the manufacturer's instructions, and where, for BUN measurement, S=6, S+P=5, PNx=6, PNx+P=5, PNx+NaKtide=5, and PNx+scramble pNaKtide=6 (*, p<0.05; **, p<0.01; ##, p<0.01 vs. PNx).

FIG. 7 is a schematic diagram illustrating the role of the Na/K-ATPase-Src signaling in the development and progression of anemia of CKD, and showing the effects of oxidative stress on RBC deformability and aging.

### BRIEF DESCRIPTION OF THE SEQUENCE LISTING

The following is a brief description of the Sequence Listing that is attached hereto and is hereby incorporated by reference in its entirety.

SEQ ID NO: 1 is an amino acid sequence of an embodiment of a polypeptide in accordance with the presently-disclosed subject matter (NaKtide);

SEQ ID NO: 2 is an amino acid sequence of a TAT cell penetrating peptide;

SEQ ID NO: 3 is an amino acid sequence of a penetratin (AP) cell penetrating peptide; and

SEQ ID NO: 4 is an amino acid sequence of the N-terminal poly-lysine domain of the al subunit of Na/K-ATPase (AiN);

SEQ ID NO: 5 is another amino acid sequence of an embodiment of a polypeptide in accordance with the presently-disclosed subject matter (pNaKtide); and

SEQ ID NO: 6 is another amino acid sequence of an embodiment of a polypeptide in accordance with the presently-disclosed subject matter (sc-pNaKtide).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

It is appreciated that the Na/K-ATPase signaling-mediated oxidant amplification loop contributes to uremic cardiomyopathy and anemia induced by ⅚^{th} partial nephrectomy (PNx) in mice, and that this process can be ameliorated by systemic administration of the peptide, pNaKtide (SEQ ID NO: 5), which was designed to block this oxidant amplification loop. It has now been surprisingly and unexpectedly discovered, however, that PNx induces anemia which was characterized by marked decreases in red blood cell (RBC) survival and number as assessed by biotinylated RBC clearance despite increases in plasma erythropoietin (EPO) concentration, eryptosis (as assessed cell surface phosphatidylserine level by Annexin V-APC binding), as well as reticulocyte count. Indeed, in these studies, no significant changes in iron homeostasis were observed, and examination of plasma samples demonstrated that PNx induced significant oxidant stress as assessed by protein carbonylation. Systemic administration of pNaKtide led to normalization of the oxidant stress, and it also normalized RBC clearance and the hematocrit without notable changes in EPO concentrations or iron metabolism. Without wishing to be bound by any particular theory or mechanism, it was thus believed that blockage of Na/K-ATPase signaling-mediated oxidant amplification loop ameliorates the anemia of experimental renal failure by increasing the RBC lifespan.

The presently-disclosed subject matter thus includes compositions and methods for treating anemia and/or increasing red blood cells (RBCs) half-life. The term “anemia”, as indicated above, is used herein to refer to a multifactorial disease process resulting in a reduction in the number of healthy RBCs that are capable of carrying adequate oxygen to the body of a subject or a reduction in the total number of circulating RBCs as evidenced by a decreased hemoglobin, hematocrit, or RBC count. Hematocrit is the percentage of the blood volume that is occupied by red blood cells or erythrocytes. In some embodiments, a method for treating anemia is provided that includes the steps of first identifying a subject as having anemia that is characterized by a reduced number of red blood cells.

In some of the presently-described methods for treating anemia and/or increasing red blood cell half-life, the methods then make use of a polypeptide that inhibits the receptor and signaling function of the Na/K-ATPase and Src complex. In some embodiments, the polypeptide is an antagonist for the receptor and signaling function of the Na/K-ATPase and Src complex. The terms “polypeptide,” “protein,” and “peptide” are used interchangeably herein to refer to a polymer of the protein amino acids regardless of its size or function. The terms “protein,” “polypeptide,” and “peptide” are used interchangeably herein to also refer to a gene product, homologs, orthologs, paralogs, fragments, any protease derived peptide (fragment), and other equivalents, variants, and analogs of a polymer of amino acids.

In some embodiments, the polypeptides are comprised of the sequence of SEQ ID NO: 1 (NaKtide), or fragments, and/or variants thereof. The terms “polypeptide fragment” or “fragment” when used in reference to such a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus of the reference polypeptide, the carboxy-terminus of the reference polypeptide, or both. Polypeptide fragments can also be inclusive of “functional fragments,” in which case the fragment retains some or all of the activity of the reference polypeptide.

The term “variant,” as used herein, refers to an amino acid sequence that is different from the reference polypeptide by one or more amino acids. In some embodiments, a variant polypeptide may differ from a reference polypeptide by one or more amino acid substitutions. For example a NaKtide polypeptide variant can differ from the NaKtide polypeptide of SEQ ID NO: 1 by one or more amino acid substitutions, i.e., mutations. In this regard, polypeptide variants comprising combinations of two or more mutations can respectively be referred to as double mutants, triple mutants, and so forth. It will be recognized that certain mutations can result in a notable change in function of a polypeptide, while other mutations will result in little to no notable change in function of the polypeptide. In this way, in some embodiments, the polypeptide variant can also be inclusive of “functional variants,” in which case the variant retains some or all of the activity of the reference polypeptide.

In some embodiments, the present polypeptides include polypeptides that share at least 75% homology with the pNaKtide polypeptide of SEQ ID NO: 1. In some embodiments, the polypeptides share at least 85% homology with the NaKtide polypeptide of SEQ ID NO: 1. In some embodiments, the polypeptides share at least 90% homology with the NaKtide polypeptide of SEQ ID NO: 1. In some embodiments, the polypeptides share at least 95% homology with the NaKtide polypeptide of SEQ ID NO: 1.

“Percent identity,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990).

Embodiments of the present polypeptides can further comprise one or more leader sequences, and in some embodiments the leader sequences include, but are not limited to, cell penetrating peptides (CPPs). The term “cell penetrating peptide” (CPP) is used herein to generally refer to short peptides that can or that assist in facilitating the transport of molecular cargo across plasma membranes found in a cell. In some instances, the molecular cargo includes another polypeptide, such as the polypeptides described herein. Of course, the cell penetrating peptides can be conjugated to the molecular cargo (e.g., polypeptide) via any number of means, including covalent bonds and/or non-covalent bonds. In a number of instances, however, such cell penetrating peptides will often include a relatively high concentration of positively-charged amino acids, such as lysine and arginine, and will have a sequence that contains an alternating pattern of charged (polar) and non-charged amino acids.

In some embodiments of the presently-disclosed subject matter, an exemplary leader sequence or cell-penetrating peptide can include the trans-activating transcriptional activator (TAT) cell penetrating peptide, which is represented by the sequence of SEQ ID NO: 2 and which when combined with the NaKtide peptide of sequence of SEQ ID NO: 1 generates a peptide designated pNaKtide referred to and described herein below. Another exemplary leader sequence includes penetratin (AP), which is represented by the sequence of SEQ ID NO: 3. Yet another exemplary leader sequence includes an amino acid sequence of the N-terminal poly-lysine domain of the al subunit of Na/K-ATPase (A1N), which is represented by the sequence of SEQ ID NO: 4. Those of ordinary skill will appreciate though that other leader sequences, including other cell penetrating peptides, can also be used in conjunction with the presently-disclosed polypeptides. In some embodiments, a polypeptide including a leader sequence, such as a cell penetrating peptide, attached to the NaKtide sequence of SEQ ID NO: 1 is referred to herein as a pNaKtide (e.g., SEQ ID NO: 5; GRKKRRQRRRPPQSATWLALSRIAGLCNRAVFQ, which includes the TAT cell penetrating peptide of SEQ ID NO: 2 fused to the NaKtide sequence of SEQ ID NO: 1).

The presently-disclosed subject matter further includes and makes use of pharmaceutical compositions comprising the polypeptides described herein as well as a pharmaceutically-acceptable carrier. Indeed, when referring to certain embodiments herein, the terms “polypeptide” and/or “composition” may or may not be used to refer to a pharmaceutical composition that includes the polypeptide.

The term “pharmaceutically-acceptable carrier” as used herein refers to sterile aqueous or nonaqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants, such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride, and the like.

Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of polypeptide to biodegradable polymer and the nature of the particular biodegradable polymer employed, the rate of polypeptide release can be controlled. Depot injectable formulations can also be prepared by entrapping the polypeptide in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations can further include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions can also take forms such as suspensions, solutions, or emulsions in oily or aqueous vehicles, and can contain formulatory agents such as suspending, stabilizing and/or dispersing agents. Alternatively, the polypeptides can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of tablets or lozenges formulated in a conventional manner.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

As described herein, the presently-disclosed subject matter further includes methods for treating anemia and/or increasing red blood cell half-life with a polypeptide. In some embodiments, the methods include administering one of the presently-disclosed polypeptides to a subject in need thereof. In some embodiments, the methods include contacting a red blood cell with one of the presently-disclosed polypeptides. The polypeptide can treat anemia and/or elongate red blood cell half-life by inhibiting the receptor and signaling function of the Na/K-ATPase and Src complex, and in some embodiments the polypeptides inhibit the receptor function by acting as an antagonist of the Na/K-ATPase and Src complex.

With respect to the treatment of anemia described herein, the terms “treatment” or “treating” are used herein to refer any treatment of anemia characterized by a reduced number of red blood cells, including, but not limited to prophylactic treatment and therapeutic treatment. As such, the terms “treatment” or “treating” include, but are not limited to: reducing the development or likelihood of development of anemia; inhibiting the progression of anemia; arresting or reducing the further development of anemia; reducing the severity of anemia; ameliorating or relieving symptoms associated with anemia; and causing a regression of the anemia or one or more of the symptoms associated with anemia.

For administration of a therapeutic composition as disclosed herein (e.g., the pNaKtide), conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the presently-disclosed subject matter include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for deterrence of a disease or condition.

Regardless of the route of administration, the polypeptide antagonists utilized in accordance with the presently-disclosed subject matter are typically administered in amount effective to achieve the desired response. As such, the term “effective amount” is used herein to refer to an amount of the therapeutic composition (e.g., a NaKtide polypeptide and a pharmaceutically vehicle, carrier, or excipient) sufficient to produce a measurable biological response (e.g., a decrease in anemia). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Florida; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pennsylvania; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

As noted, in some embodiments of the presently-disclosed subject matter, treating the anemia or increasing the half-life of red blood cells comprises administering the polypeptide antagonist in an amount sufficient to treat one or more of the symptoms or underlying causes or symptoms of the anemia or the half-life of the red blood cells. For instance, in some embodiments, the polypeptide antagonist is administered in amount sufficient to increase a hematocrit level in the subject, to reduce an amount of reticulocytes in the subject, or to decrease an amount of eryptosis in the subject. In some embodiments, the polypeptide antagonist is administered in amount sufficient to increase a half-life of a red blood cell, including the half-life of a red blood cell in a subject.

Various methods known to those skilled in the art can be used to determine an increase or a reduction in such factors and symptoms associated with anemia or red blood cell half-life in a subject. For example, in certain embodiments, an increase in red blood cell half-life can be measured by labeling an amount of red blood cells at a first time point and then detecting the amount of the labeled cells at one or more later time points when the red blood cells have cleared from the blood stream. The half-life of the red blood cells or, in other words, the time it takes for the red blood cell concentration to reach one-half of its steady state value can then be calculated for a particular subject.

With respect to the increases in hematocrit levels, reductions in an amount of reticulocytes, decreases in an amount of eryptosis, and the like, the terms “increase” or “decrease” or “reduction” or grammatical variations thereof do not necessarily refer to the ability to completely activate or completely inactivate all target biological activity in all cases. Rather, the skilled artisan will understand that such increases or decreases in biological activity or inactivity can be determined relative to a control, wherein the control can be representative of an environment, for example, in which a therapeutic agent (e.g., a polypeptide antagonist) is not administered or is administered at a fixed or other amount. For example, in some embodiments, an increase or decrease in activity or inactivity relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% increase or decrease.

With still further regard to the various methods described herein, although certain embodiments of the methods disclosed herein only call for a qualitative assessment, other embodiments of the methods call for a quantitative assessment. Such quantitative assessments can be made, for example, using known methods, as will be understood by those skilled in the art.

The skilled artisan will also understand that measuring a reduction, increase, or other change in biological activity or inactivity is a statistical analysis. For example, an increase in red blood cell half-life can be compared to a control level of red blood cell half-life (e.g., a red blood cell half-life observed in subjects that are not anemic), and a red blood cell half-life greater than or equal to the control level can be indicative of an increase in red blood cell half-life, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like. In some embodiments, the subject is a male subject.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. The following examples may include compilations of data that are representative of data gathered at various times during the course of development and experimentation related to the present invention.

### EXAMPLES

### Materials and Methods

Chemicals, reagents, and antibodies: All chemicals, except otherwise mentioned, were obtained from Sigma-Aldrich (St. Louis, MO). EZ-Link Sulfo-NHS Biotin (water-soluble and cell membrane-impermeable, Cat #A39256), fluorescent DNA dye 7-Aminoactinomycin-D (7-AAD, Cat #A1310), Thiazole Orange (Cat #50-196-4656), protease/phosphatase inhibitor cocktail (Cat #78442), and Pierce Reversible Protein Stain Kit—for PVDF membrane (Cat #24585) were from ThermoFisher Scientific (Waltham, MA). Antibody against EPO (clone B-4, Cat #sc-5290), RIPA lysis buffer system (Cat #sc-24948), and HRP-conjugated secondary antibodies were from Santa Cruz (Santa Cruz, CA). Streptavidin-PE (Cat #562284), Annexin V binding buffer (10×, Cat #556454), Annexin V-APC (Cat #550475), and microtainer (Cat #365965) were from BD Biosciences (San Jose, CA). DNPH (Cat #D199303) and anti-DNP antibody (Cat #D9656) were from Sigma-Aldrich. Urea assay kit (Cat #ab8362) for plasma BUN assay was from Abcam (Boston, MA), and Iron/TIBC Reagents (Cat #1750460) was from Pointe Scientific (Canton, MI).

Animal study: All animal care and experiments were approved by the Marshall University Institutional Animal Care and Use Committee (IACUC) in accordance with the National Institutes of Health (NIH) Guide for the Care and Use of Laboratory Animals. The animal facility is AAALAC-accredited and maintained at an SPF status. C57BL6 mice (10-12 weeks old) were purchased from the Jackson Laboratory (Bar Harbor, ME) and housed in a pathogen-free animal facility under a 12-hour light/dark cycle. Standard rodent chow diet (Rodent Diet 5001, LabDiet, St Louis, MO) and water were supplied ad libitum. Mice were randomly divided into six experimental groups: (1) Sham; (2) Sham+pNaKtide; (3) ⅚th partial nephrectomy (PNx); (4) PNx+pNaKtide; (5) PNx+NaKtide; and (6) PNx+scramble pNaKtide (sc-pNaKtide).

The ⅚^{th} partial nephrectomy with pole ligation (PNx) and sham surgery: PNx and sham surgery methods were performed as described previously. Briefly, the PNx surgery is divided into two separate procedures. The first surgery involves ligation of the left kidney of the upper and lower poles. Seven days later, the second surgery involves the removal of the right kidney. If the mice were in overall healthy condition after 3 days, the study was commenced. For Sham surgery, the same surgery procedures were performed, including exposure of the kidney, dissection of tissue, and wound closure, but without the left kidney's pole ligation and removal of the right kidney. This PNx model has shown uremic cardiomyopathy and anemia.

pNaKtide, NaKtide, and scramble pNaKtide (sc-pNaKtide) injection: pNaKtide was injected subcutaneously (25 mg/kg body weight, in normal saline) one week after right kidney removal and then repeated weekly for a total of three doses. NaKtide is a 20-amino acid sequence (SATWLALSRIAGLCNRAVFQ; SEQ ID NO: 1) of the Na/K-ATPase al subunit that binds to the c-Src kinase domain to prevent c-Src Tyr416 phosphorylation. NaKtide was further utilized with an HIV-TAT leader sequence (GRKKRRQRRRPPQSATWLALSRIAGLCNRAVFQ; SEQ ID NO: 5) to make the cell membrane permeable pNaKtide and to keep pNaKtide to the intracellular face of the cell membrane, which largely limited the effects of pNaKtide to membrane-associated (including Na/K-ATPase-associated) c-Src. It has been shown that the TAT-fusion proteins may have nuclear effects and may get trapped in endosomes and/or lysosomes. To test the effect of the TAT leader sequence, one PNx group was administrated with a scrambled pNaKtide (sc-pNaKtide; GRKKRRQRRRPPQACWIQNLSRSAGATVRLFLA; SEQ ID NO: 6). To test if cell membrane-impermeable NaKtide has a similar effect as membrane-permeable pNaKtide, another PNx group was administrated with NaKtide.

Assessment of RBC half-life by flow cytometry: The RBC half-life was assessed by labeling RBC with biotin and detection with streptavidin-PE. EZ-Link Sulfo-NHS Biotin was dissolved in sterile 1×PBS and passed through a 0.22-μm sterilized filter before use. Mice received the EZ-Link Sulfo-NHS biotin solution via cardiothoracic puncture at a dose of 1.0 mg biotin/25 g body weight, 3-days post-second surgery. Bodyweight for each mouse was measured to calculate the dose and volume. The total volume administered per mouse was no greater than 7.5% of the circulating blood volume (assuming 1.46 ml of a mouse of 25 g body weight) to limit possible suppression of normal erythropoiesis.

The first blood samples were obtained 3-days post biotin injection (designed as Day 3). The blood samples were then obtained on Day 7, 14, 21, and 28 post-biotin injection. The whole blood samples were collected into BD microtainer tubes coated with lithium Heparin from the submandibular vein. 10 μL of the whole blood sample was transferred to pre-chilled microcentrifuge tubes containing 200 μL mixture of 1×PBS with anticoagulant acid citrate-dextrose solution A (ACD-A) (PBS/ACD-A, 1:4, vol/vol). ACD-A solution was prepared according to United States Pharmacopeia (USP) formula. 10 μL of Streptavidin-PE (0.5 mg/mL) was gently mixed with each blood sample and incubated in the dark for 10 min at room temperature. The samples were then washed twice with 400 μL PBS/ACD-A each by centrifugation (500×g for 15 min at 4° C.). The pellet was resuspended in 200 μL PBS/ACD-A and analyzed by flow cytometry (NovoCyte 3000 Flow Cytometer Systems, Agilent Technologies, Santa Clara, CA). Each sample was processed in triplicate for the flow cytometry study. Data was analyzed with NovoExpress software v1.3 with positive signal gating based upon negative (unlabeled) controls.

Assessment of cell surface Phosphatidylserine (PS) level of RBC by flow cytometry: The PS-specific binding of Annexin V was used to quantitate PS on RBC membrane for eryptosis. Annexin V is a member of the annexin family of calcium-dependent phospholipid-binding proteins with a high affinity for PS. For flow cytometry analysis, 10 μl of Annexin V-APC was added to 200 uL of diluted blood sample (1:40, vol/vol, with pre-chilled 1× Annexin-V binding buffer) and incubated for 15 min at room temperature in the dark. Each sample was prepared in triplicate. Fifty thousand events per sample were acquired on the Novocyte 3000 Flow Cytometer. Fluorescence parameters were collected and analyzed using NovoExpress software v1.3. The RBC population was defined by size in forward and side scatter plots, and the gated cells were counted as Annexin V-positive if they had a fluorescence greater than the negative control. Annexin V-APC binding to RBCs was given as a percentage of Annexin V-positive cells. The threshold value was determined in preliminary studies based on analysis of the negative and positive controls. For negative controls, samples were processed with Annexin V-APC in 1× Annexin V binding buffer without CaCl₂) and 1× Annexin V binding buffer with CaCl₂) but without the Annexin-V APC. For positive controls, samples were processed with Tween-20 (0.01%, vol/vol) in 1× Annexin V binding buffer plus Annexin-V APC. 7-AAD was used to exclude permeabilized and dead cells. Each sample was processed in triplicate.

Assessment of reticulocyte count by flow cytometry: To measure the reticulocyte count, 10 μl of whole blood sample was gently mixed with 200 μl of pre-chilled PBS/ACD-A (1:4, vol/vol) solution, and then incubated with 10 μl of Thiazole Orange at room temperature in the dark for 30 minutes. The samples were washed twice with 400 μl of PBS/ACD-A buffer each (500×g for 15 minutes, 4° C.). Pellets were then suspended in 200 μl of PBS/ACD-A buffer and analyzed with the flow cytometer. The erythrocyte population was defined by software evaluation of FSC and SSC parameters, thus excluding most nucleated cells and platelets. The cells of interest were then evaluated for fluorescence characteristics. Gated populations were set between the negative and positive populations in the fluorescence histogram for Thiazole Orange, thus distinguishing between reticulocytes and mature erythrocytes. The reticulocytes were expressed as percentages of the total erythrocytes. Each sample was processed in triplicate.

Assessment of iron homeostasis: Plasma iron concentration, TIBC (total iron-binding capacity), UIBC (unsaturated iron-binding capacity), and iron saturation rate (%) were assessed with Iron/TIBC Reagent Set (Cat #1750460) from Pointe Scientific (Canton, MI), according to the manufacturer's instruction.

Immunoblotting analyses of protein carbonylation and EPO in plasma samples: Plasma (50 μL) samples were lysed with 250 μL of RIPA lysis buffer (with protease and phosphatase inhibitor cocktail) and cleared by centrifugation. The protein concentration was determined by BCA protein assay kit (Bio-Rad). For protein carbonylation analysis, equal amounts of total protein from each sample were denatured with 6% SDS (final concentration), derivatized with 1×DNPH (freshly diluted with distilled water from 10×DNPH stock solution, 100 mM in 100% trifluoroacetic acid) to form DNP hydrazone derivative, and then neutralized with neutralization buffer (30% of glycerol in 2 M Tris). For plasma EPO analysis, equal amounts of total protein from each sample were used. The samples were separated with Novex 10% Tris-Glycine gel (Cat #XP00100, Invitrogen, ThermoFisher Scientific). After transfer proteins to a PVDF membrane, the membrane was stained with Pierce Reversible Protein Stain Kit—for PVDF membrane for protein loading control, followed by immunoblotting analysis with anti-DNP antibody for protein carbonylation or anti-EPO antibody for plasma EPO. The signals from immunoblotting and PVDF membrane protein stain were obtained with FluorChem M and calculated with AlphaView software (ProteinSimple).

Statistical analysis: Data were tested for normality and then subjected to parametric analysis. Statistical significance was reported at the P<0.05 and P<0.01 levels. RBC ½ life was calculated by performing a least squares linear regression on the log 2 of the fraction of biotinylated RBCs vs. time. The open-source R program was used for all data analyses and graph preparations as before.

### Example 1—PNx Shortened RBC Half-Life and pNaKtide Corrected Anemia

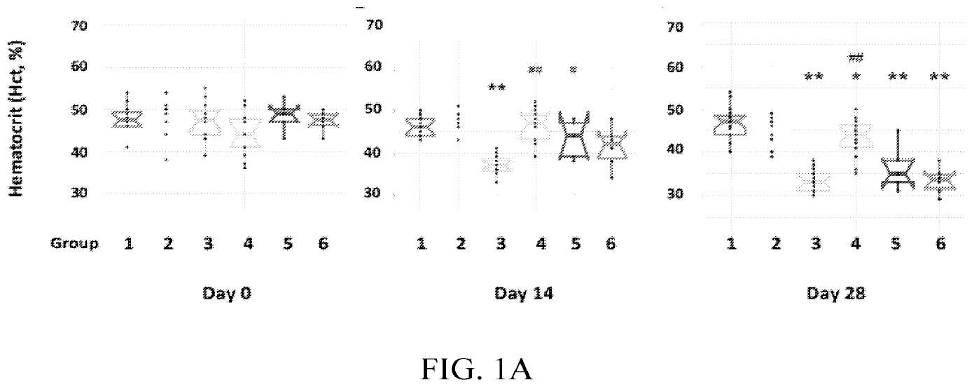

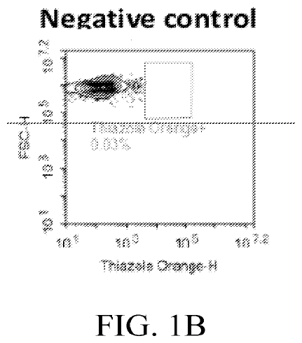

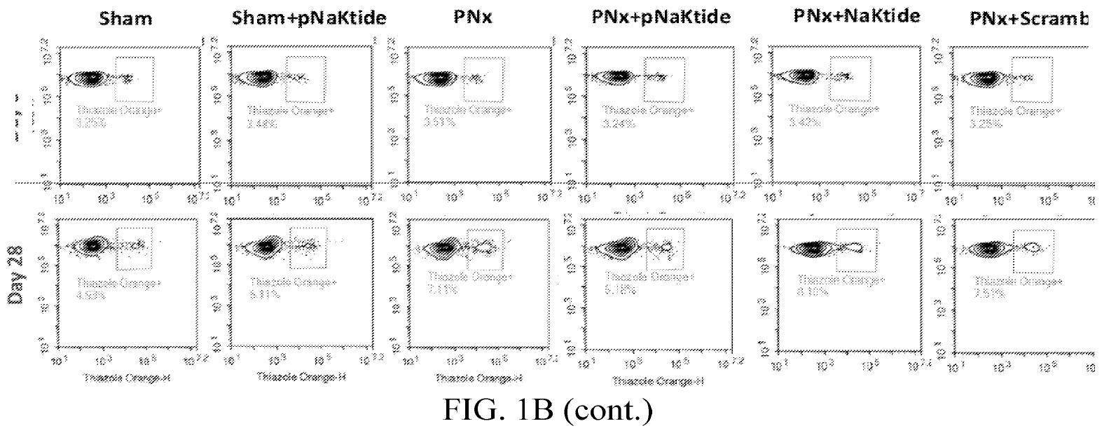

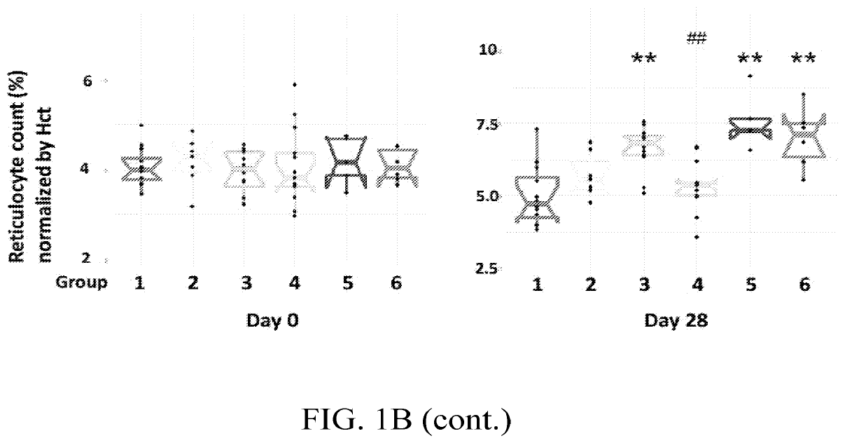

Hematocrit values were significantly reduced in animals with experimental renal failure compared with the Sham group. Concomitant administration of pNaKtide ameliorated the anemia whereas pNaKtide administration to sham animals did not change hematocrit values (FIG. 1A). On the other hand, at the end of the experiment, reticulocyte count (normalized with hematocrit) was significantly higher in PNx group compared with the sham group. However, administration of pNaKtide did not significant affect reticulocyte count in the Sham group but significantly reduces reticulocyte count in PNx group. Administration of NaKtide or sc-pNaKtide did not affect reticulocyte count in the PNx group (FIG. 1B).

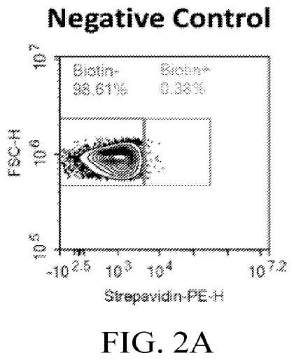

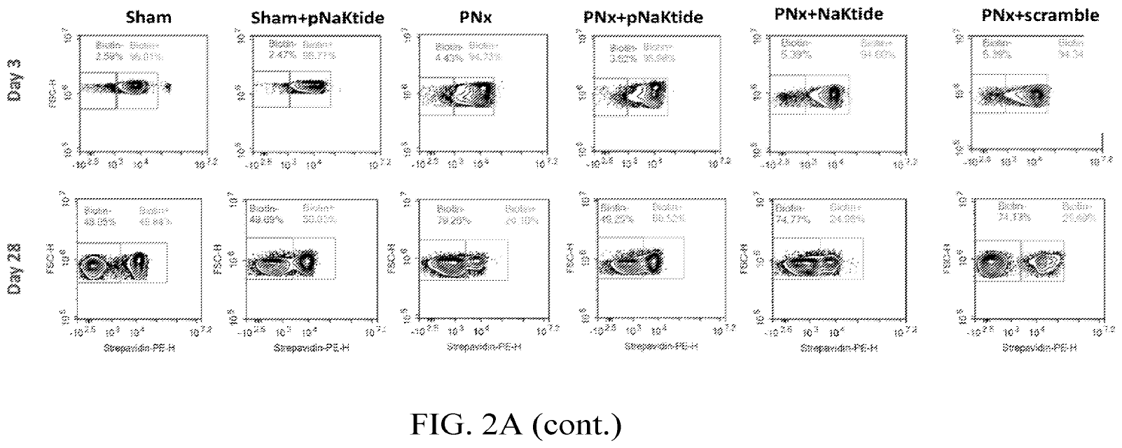

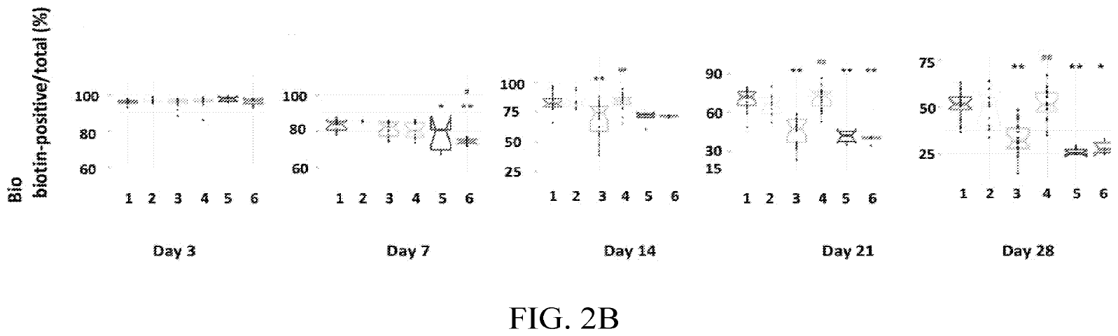

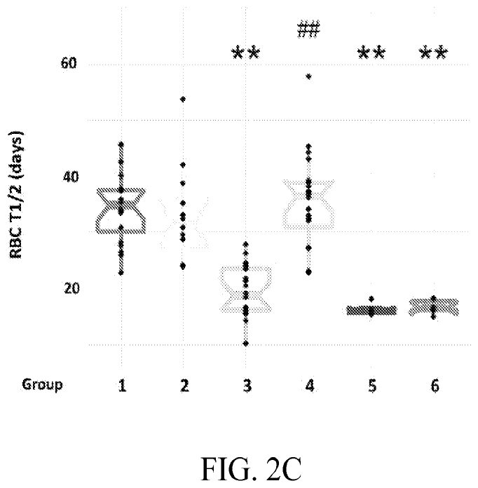

Using the biotinylated RBC method, an RBC half-life of 34 days was seen in the sham group which was reduced to 19 days in the PNx group. While the Sham+pNaKtide group showed a half-life of 32 days and the PNx+pNaKtide group showed a half-life of 34 days, there was no significant increase in RBC half-life in PNx groups treated with NaKtide or sc-pNaKtide compared with the PNx group (FIG. 2). These data, especially from the PNx group, indicated that amplified generation of ROS were an explanation for inducing anemia in chronic kidney disease (CKD). The effect of pNaKtide showed that reversal of anemia is possible by blocking the Na/K-ATPase-Src signaling pathway by preventing the amplified generation of ROS.

### Example 2—PNx and pNaKtide Administration Did not Significantly Change Iron Homeostasis and Plasma EPO Level

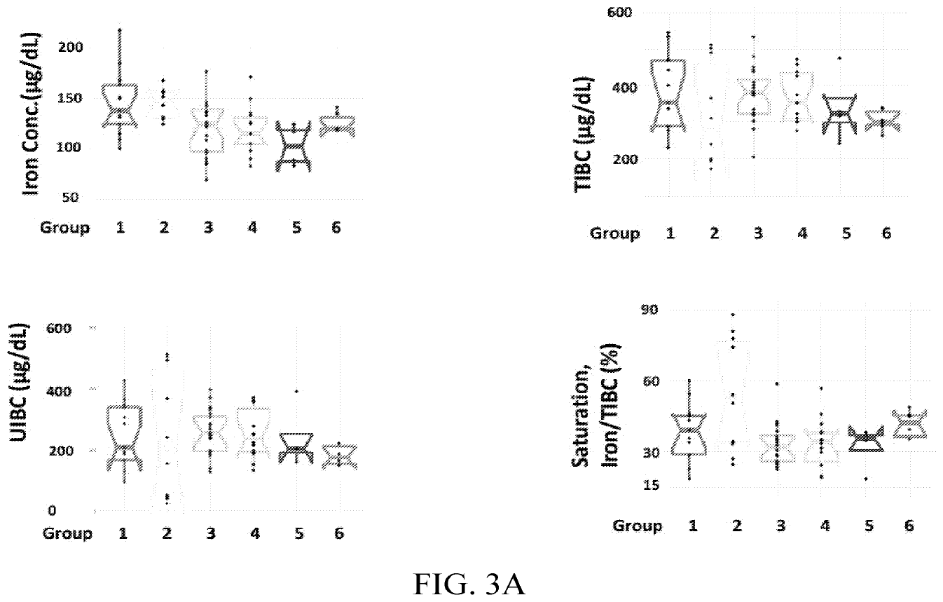

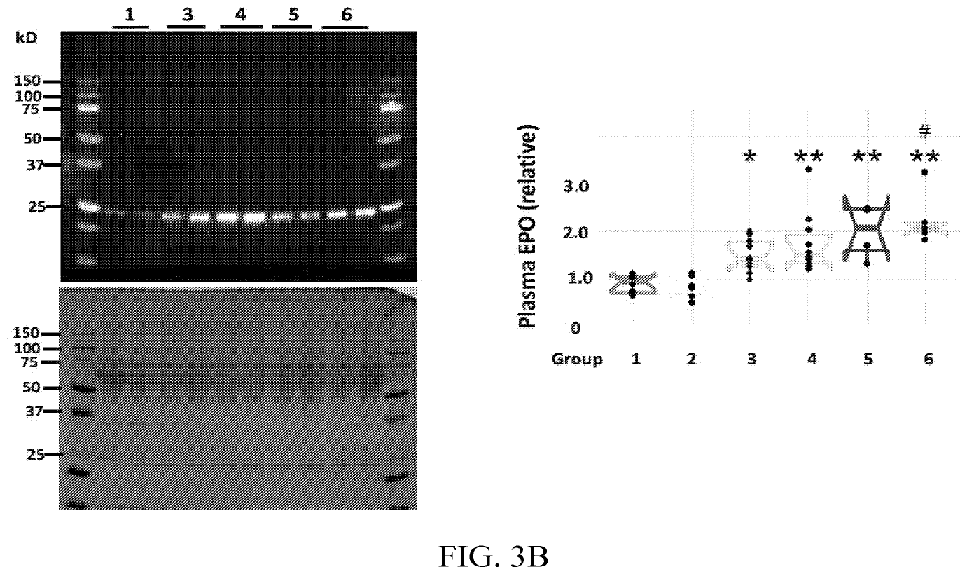

At the end of the experiment, there were no significant differences concerning plasma iron level, TIBC, UIBC, and iron saturation rate (FIG. 3A). Furthermore, plasma EPO levels in the Sham and Sham+pNaKtide groups were similar, but the EPO levels in the PNx group and PNx groups treated with pNaKtide, NaKtide, or sc-pNaKtide are significantly higher compared with the Sham group (FIG. 3B). These data indicated that PNx-mediated anemia might not involve the disturbance of iron homeostasis and EPO deficiency.

### Example 3—PNx-Stimulated Eryptosis Corrected by pNaKtide Administration

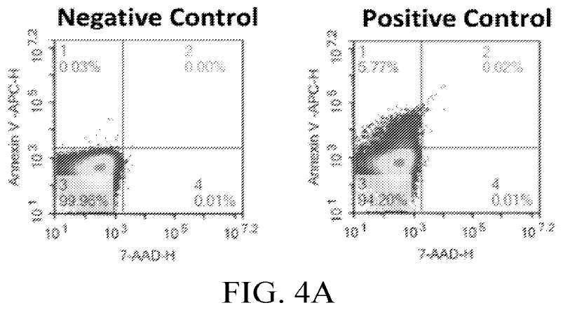

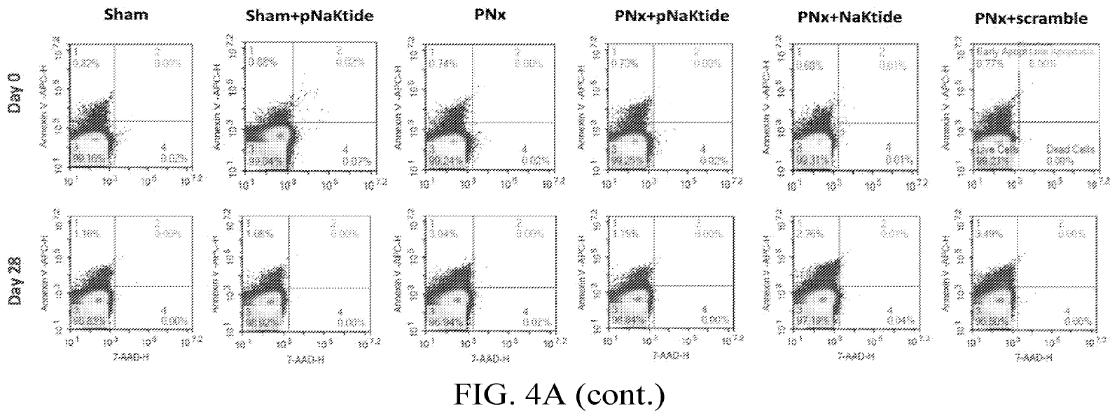

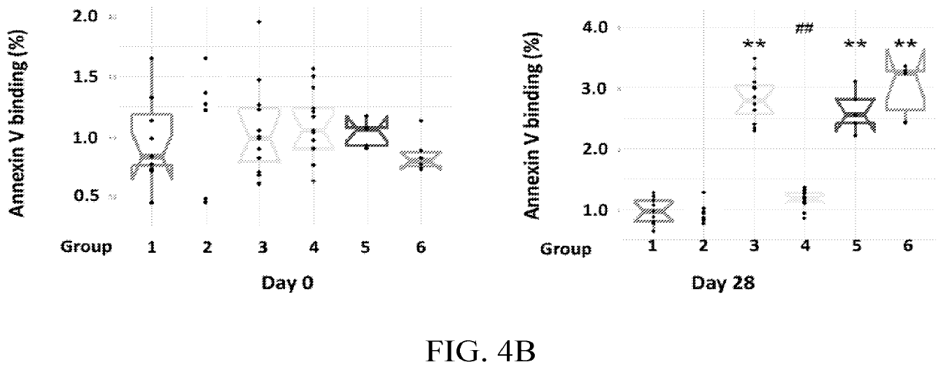

Annexin V binding to externalized PS is a well-documented marker of eryptosis (programmed suicidal RBC death). Blood samples were collected at Day 0 (before Sham and PNx surgery as baseline) and Day 28 post surgery. On Day 0, similar baseline values were observed across six groups. On Day 28, only the PNx group showed a significant increase in Annexin V binding (increase in PS exposure at the cell surface) that was blocked by pNaKtide, but not by NaKtide or sc-pNaKtide administration (FIG. 4). The data indicate that PNx-induced anemia can be attributed to the higher rate of eryptosis than the new RBC formation rate (see FIG. 1B, Reticulocyte count).

### Example 4—PNx Increased Protein Carbonylation of Plasma Samples Corrected by pNaKtide Administration

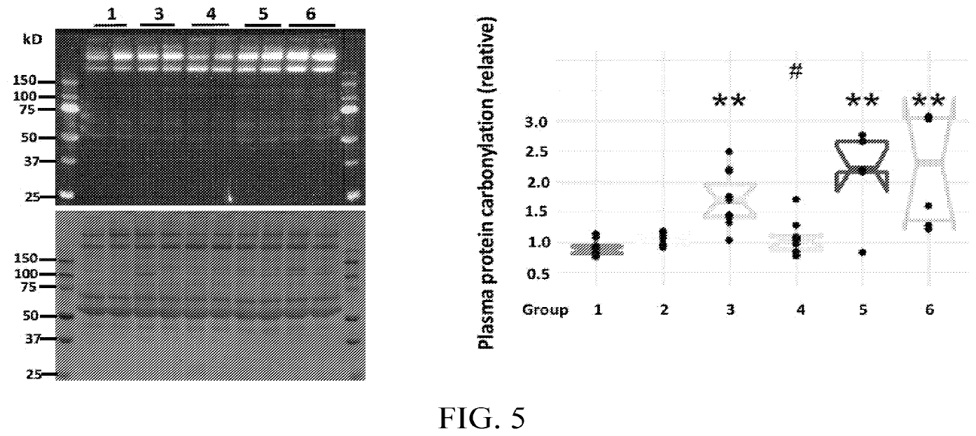

It has been shown that PNx activates Na/K-ATPase signaling mediated oxidant amplification loop and increase protein carbonylation modification in different tissues that was ameliorated by pNaKtide administration. To further evaluate the bloodstream's oxidative status, which will affect RBCs more directly, protein carbonylation, a protein oxidation marker, was measured in the plasma samples obtained at the experiment's conclusion. As shown in FIG. 5, the protein carbonylation modification level is significantly higher in PNx group than in the Sham group, and pNaKtide administration reduces PNx-induced protein carbonylation nearly back to normal level. However, administration of NaKtide or sc-pNaKtide in the PNx groups showed higher carbonylation modification as seen in the PNx group. Since increased oxidative stress is an important contributor to RBC eryptosis, aging, and deformability, the data further indicate the role of Na/K-ATPase signaling mediated oxidant amplification loop in PNx-induced anemia.

### Example 5—PNx Impaired Renal Function not Corrected by pNaKtide Administration

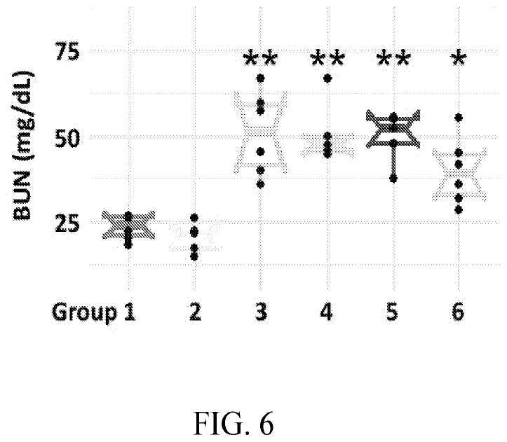

Plasma BUN and creatinine levels were measured to assess the effect of PNx (and administration of the pNaKtide, NaKtide, and sc-pNaKtide) on renal function. The PNx group showed significantly increased plasma BUN and creatinine compared with the Sham group. Administration of pNaKtide, NaKtide, and sc-pNaKtide in PNx groups did not ameliorate PNx-induced increase in BUN (FIG. 6). For plasma creatinine level measured with mouse creatinine assay Kit (Cat #80350, Crystal Chem Inc.), PNx induced significant increase in creatinine level compared with the Sham group, and administration of pNaKtide in PNx mice did not significantly reduce PNx-induced increase in creatinine level compared to the PNx group (data not shown). These results largely agree with previous observations that, neither systemic administration of pNaKtide nor lentiviral specific delivery of NaKtide to adipocytes resulted in substantial changes in plasma BUN and creatinine in the setting of PNx.

### Discussion of Examples 1-5

CKD may be caused by many risk factors, including diabetes, hypertension, heart disease, hereditary causes, race, and age, especially over 60. The relationship between CKD and anemia was first discovered over 170 years ago by Dr. Richard Bright in the 1800s. Since its discovery, the causes of CKD have been found to be multifactorial. It was found that in circulation, EPO levels were normal to slightly increased in anemia-related CKD. However, this was still considered inappropriately low as anemic patients with normal kidney function have about 10 to 100× higher EPO levels. PNx stimulates the Na/K-ATPase/Src signaling and oxidative stress, and other changes, leading to fibrosis in heart left ventricle and kidney. The stimulated renal fibrosis and oxidative stress might change the responses of the renal EPO-producing cells (REPs) that simultaneously stimulate renal fibrosis and erythropoiesis in the kidney. Moreover, both epidermal deletion of the von Hippel-Lindau (VHL) factor and tubular-specific deletion of VEGFA (vascular endothelial growth factor A) significantly upregulate renal EPO production. Administration of pNaKtide reduced the expression of VEGFA in human tumor cell lines that might increase EPO production.

Other studies have shown that another possible explanation may be due to uremic induced inhibitors of erythropoiesis, but no specific inhibitors have yet been discovered. CKD has also been linked to iron losses in humans. It is estimated that there is about a loss of 1-3 grams per year in CKD patients, especially in hemodialysis patients. In addition to iron losses, CKD patients have also been shown to have impaired dietary absorption of iron and functional iron deficiency.

More importantly related to the above-described study, there have been studies showing shortened RBC survival in CKD patients. In humans, it has been shown that the RBC half-life span in these patients shortened to approximately half the normal lifespan. Possible explanations for decreased RBC survival include contributions of the toxic uremic environment and reduced number of Na/K-ATPase on the RBC membrane. The shortened RBC lifespan was also attributed to interference of an unidentified substance in uremic plasma relating to the function of Na/K-ATPase. With CKD-induced anemia being multifactorial and not yet fully understood, the present studies offered more insight on other possible explanations of CKD-induced anemia and specifically focusing on ROS and the Na/K-ATPase. The major source of iron for erythropoiesis is the recycling of iron from senescent RBCs.

In the presently-described study, it was found that the ⅚th PNx induced anemia in C57BL/6 mice may not be due solely to iron deficiency anemia and instead due to the generation of ROS. Through biotin-labeled RBC and the measurement through flow cytometry detecting biotin-streptavidin-PE complex, the studies were able to show improvement in the half-life of RBC in the Sham+pNaKtide and PNx+pNaKtide groups (FIG. 2). Prior studies have shown that oxidative stress is a stimulator of eryptosis and contributes to the development of anemia. In prior mice studies, it was shown that iron deficiency is not the sole explanation for inducing anemia. The present results support that generation of ROS and the reversal via pNaKtide can be an alternate explanation to those findings (FIG. 5). Other RBC studies in mice have shown a half-life between male and female C57BL/6 mice (22.9±1.2 and 22.4±0.9 d, respectively). The present study showed a half-life of 34 in the sham group and 19 in the PNx group. The pNaKtide+Sham group increased half-life life to 33 days and in the PNx+pNaKtide group to 35 days (FIG. 2). These data, especially in the PNX group, indicated that generation of ROS can be an explanation for inducing anemia in CKD and that the pNaKtide showed that reversal of anemia was possible by blocking the Na/K-ATPase-Src signaling pathway. To further support the hypothesis, flow cytometry looking at Annexin V binding levels was performed. A cell's plasma membrane contains a lipid called phosphatidylserine (PS) which is normally found in the inner leaflet of the plasma membrane on the cytoplasmic side. During apoptosis and eryptosis, PS will be exposed to the outer leaflet of the plasma membrane instead of being confined to the cytoplasmic side. Annexin V is a 36-kDa calcium binding protein which directly binds to PS. Therefore, by using Annexin V binding assay, it was possible to measure and detect apoptotic cells. What was seen was a significant increase in Annexin V binding levels that was corrected by pNaKtide administration (FIG. 4), while a simultaneous decrease in biotin-positive percentages over time (FIG. 2).

Anemia of CKD is generally characterized by iron deficiency and EPO deficiency, but clinical treatment with iron supplementation and erythropoiesis-stimulating agents (ESAs) could only partially reverse anemia status, indicating other mechanisms. Accumulating evidence indicates that anemia of CKD is mainly the result of accelerated eryptosis. Eryptosis is a process of multifactor-induced programmed suicidal RBC death that occurred ahead of normal RBC senescence to eliminate infected or defective RBCs. In CKD patients, enhanced eryptosis leads to early and accelerated RBC clearance, stimulated by factors such as uremic toxins, ROS, and pro-inflammatory cytokines. Since eryptotic cells are rapidly cleared, anemia happened when the rate of eryptosis exceeds the rate of erythropoiesis. The increased number of eryptotic RBCs is closely related to a shortened RBC lifespan which further compounds renal anemia. Moreover, CKD progression was associated with shortened RBC lifespan in CKD patients but not directly correlated with serum EPO, ferritin, or vitamin B12.

In the pole-ligation ⅚th PNx mouse model, there are substantial increases in ROS, protein carbonylation modification, pro-inflammatory cytokines, and uremia toxins. Interestingly, according to the reticulocyte count (FIG. 1B) and annexin V binding assays (FIG. 4), both eryptosis and erythropoiesis appear to be accelerated in the PNx group compared with the Sham group. However, while administration of pNaKtide significantly reduces eryptosis of PNx animals, pNaKtide fails to further enhance erythropoiesis in PNx animals (FIG. 1B, reticulocyte count) even though the plasma EPO levels in both PNx and PNx+pNaKtide groups are significantly higher than the Sham group (FIG. 3B). This agrees with the concept that increased reticulocytosis may also serve as an indicator of accelerated eryptosis despite normal RBC counts. It is worth noting that, compared with baseline, reticulocyte counts of Sham and Sham+pNaKtide are both higher at the end of experiments, which might be caused by erythropoiesis stimulated by the two-step surgeries as well as regular collections of blood samples for analysis.

While an increase in ROS is a critical stimulator of eryptosis in CKD patients due to the increased ROS generation in RBCs, ROS also contributed to RBC deformability and aging, in which the Na/K-ATPase plays an important role. Moreover, uremic toxins, such as indoxyl sulfate, were shown to stimulate eryptosis and ROS generation through Na/K-ATPase signaling. This redox imbalance further results in pro-inflammatory cytokine production that positively correlates with increasing stages of CKD. Decrease in the number and activity of Na/K-ATPase and increase in ROS are two significant contributors in RBC aging and shortened RBC lifespan. Na/K-ATPase was significantly decreased not only by aging, but also in CKD patients with end-stage renal failure on dialysis and type 2 diabetes mellitus as well. The Na/K-ATPase al subunit and tyrosine kinase c-Src also form a redox-sensitive signaling complex that can be activated by both CTS and ROS, leading to the amplification of ROS generation in a feed-forward mechanism that was involved in PNx-mediated uremic cardiomyopathy and anemia. Inhibition of Na/K-ATPase signaling by pNaKtide restores systemic redox imbalance and attenuates the release of pro-inflammatory cytokines.

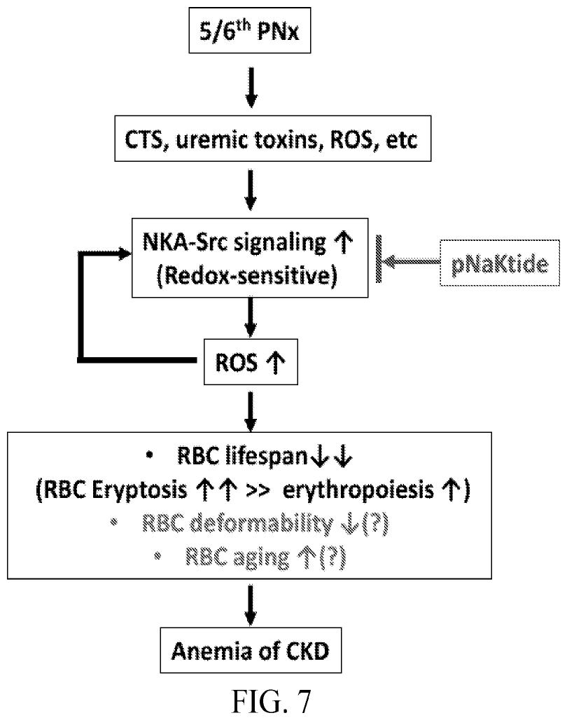

The present study supports the hypothesis that the Na/K-ATPase signaling mediated oxidant amplification loop, which shortened RBC lifespan through enhanced eryptosis, RBC deformability, and aging, plays an important role in the development of anemia in CKD. In this regard, and without wishing to be bound by any theory or mechanism, it was believed that the pole-ligation ⅚th PNx mouse model increase cardiotonic steroids, uremic toxins, oxidative stress, and other factors. These changes activate the redox-sensitive Na/K-ATPase-Src signaling and the oxidant amplification loop (which can be ameliorated by administration of pNaKtide, but not NaKtide or sc-pNaKtide), leading to anemia by shortened RBC lifespan (eryptosis rate is greater than erythropoiesis rate), decreased RBC deformability, and accelerated RBC aging (see FIG. 7).

In summary, the foregoing studies demonstrate a novel and important finding that the NKA-Src signaling mediated oxidant amplification is behind an oxidative stress-centered development and progression of anemia of CKD, without significant imbalance of iron homeostasis. This is an extension of clinical observations as well as early investigations of recovery of human RBCs' Na/K-ATPase activity/turnover rate after dialysis of CKD patients, which suggests the potential role of cardiotonic steroids, uremic toxins, and oxidative stress. In a murine model of CKD which is complicated by anemia, blockade of the Na/K-ATPase signaling with a specific peptide (pNaKtide) ameliorates the anemia primarily by increasing RBC survival. Again without wishing to be bound by any particular theory or mechanism, this strategy can allow for novel and additive strategies to treat anemia.

Throughout this document, various references are mentioned. All such references are incorporated herein by reference, including the references set forth in the following list:

### REFERENCES

1. Abed M, Artunc F, Alzoubi K, Honisch S, Baumann D, Foller M, and Lang F. Suicidal erythrocyte death in end-stage renal disease. J Mol Med (Berl) 92: 871-879, 2014.
2. Ahmed M S, Langer H., Abed M, Voelkl J, and Lang F. The uremic toxin acrolein promotes suicidal erythrocyte death. Kidney Blood Press Res 37: 158-167, 2013.
3. Aperia A. New roles for an old enzyme: Na,K-ATPase emerges as an interesting drug target. J Intern Med 261: 44-52, 2007.
4. Babitt J L, and Lin H Y. Mechanisms of Anemia in CKD. Journal of the American Society of Nephrology 23: 1631-1634, 2012.
5. Bagrov A Y, and Shapiro J I. Endogenous digitalis: pathophysiologic roles and therapeutic applications. Nat Clin Pract Nephrol 4: 378-392, 2008.
6. Bagrov A Y, Shapiro J I, and Fedorova O V. Endogenous cardiotonic steroids: physiology, pharmacology, and novel therapeutic targets. Pharmacol Rev 61: 9-38, 2009.
7. Bartlett D E, Miller R B, Thiesfeldt S, Lakhani H V, Khanal T, R D P, Cottrill C L, Klug R L, Adkins N S, Bown P C, Nease D B, Shapiro J I, and Sodhi K. Uremic Toxins Activates Na/K-ATPase Oxidant Amplification Loop Causing Phenotypic Changes in Adipocytes in In Vitro Models. Int J Mol Sci 19: 2018.
8. Batchelor E K, Kapitsinou P, Pergola P E, Kovesdy C P, and Jalal D I. Iron Deficiency in Chronic Kidney Disease: Updates on Pathophysiology, Diagnosis, and Treatment. J Am Soc Nephrol 31: 456-468, 2020.
9. Bissinger R, Bhuyan A A M, Qadri S M, and Lang F. Oxidative stress, eryptosis and anemia: a pivotal mechanistic nexus in systemic diseases. FEBS J 286: 826-854, 2019.
10. Boas F E, Forman L, and Beutler E. Phosphatidylserine exposure and red cell viability in red cell aging and in hemolytic anemia. Proceedings of the National Academy of Sciences 95: 3077-3081, 1998.
11. Bogdanova A, and Lutz H. Mechanisms tagging senescent red blood cells for clearance in healthy humans. Frontiers in Physiology 4: 2013.
12. Bonan N B, Steiner T M, Kuntsevich V, Virzi G M, Azevedo M, Nakao L S, Barreto F C, Ronco C, Thijssen S, Kotanko P, Pecoits-Filho R, and Moreno-Amaral A N. Uremic Toxicity-Induced Eryptosis and Monocyte Modulation: The Erythrophagocytosis as a Novel Pathway to Renal Anemia. Blood Purif 41: 317-323, 2016.
13. Bosch R J, Hernando N, Casado S, and Lopez-Novoa J M. Endogenous digoxin-like immunoreactivity and erythrocyte sodium transport in uraemic patients undergoing dialysis. Clin Sci (Lond) 76: 157-163, 1989.
14. Boutin A T, Weidemann A, Fu Z, Mesropian L, Gradin K, Jamora C, Wiesener M, Eckardt K U, Koch C J, Ellies L G, Haddad G, Haase V H, Simon M C, Poellinger L, Powell F L, and Johnson R S. Epidermal sensing of oxygen is essential for systemic hypoxic response. Cell 133: 223-234, 2008.
15. Bright R. Cases and Observations Illustrative of Renal Disease, Accompanied with the Secretion of Albuminous Urine. Med Chir Rev 25: 23-35, 1836.
16. Chauhan A, Tikoo A, Kapur A K, and Singh M. The taming of the cell penetrating domain of the HIV Tat: myths and realities. J Control Release 117: 148-162, 2007.
17. Cheng J T, Kahn T, and Kaji D M. Mechanism of alteration of sodium potassium pump of erythrocytes from patients with chronic renal failure. J Clin Invest 74: 1811-1820, 1984.
18. Daenen K, Andries A, Mekahli D, Van Schepdael A, Jouret F, and Bammens B. Oxidative stress in chronic kidney disease. Pediatr Nephrol 34: 975-991, 2019.
19. Dholakia U, Bandyopadhyay S, Hod E A, and Prestia K A. Determination of RBC Survival in C57BL/6 and C57B/6-Tg(UBC-GFP) Mice. Comp Med 65: 196-201, 2015.
20. Dias G F, Bonan N B, Steiner T M, Tozoni S S, Rodrigues S, Nakao L S, Kuntsevich V, Pecoits Filho R, Kotanko P, and Moreno-Amaral A N. Indoxyl Sulfate, a Uremic Toxin, Stimulates Reactive Oxygen Species Production and Erythrocyte Cell Death Supposedly by an Organic Anion Transporter 2 (OAT2) and NADPH Oxidase Activity-Dependent Pathways. Toxins (Basel) 10: 2018.
21. Dias G F, Grobe N, Rogg S, Jorg D J, Pecoits-Filho R, Moreno-Amaral A N, and Kotanko P. The Role of Eryptosis in the Pathogenesis of Renal Anemia: Insights From Basic Research and Mathematical Modeling. Front Cell Dev Biol 8: 598148, 2020.
22. Diederich L, Suvorava T, Sansone R, Keller T C S, Barbarino F, Sutton T R, Kramer C M, LuckstAdt W, Isakson B E, Gohlke H, Feelisch M, Kelm M, and Cortese-Krott M M. On the Effects of Reactive Oxygen Species and Nitric Oxide on Red Blood Cell Deformability. Frontiers in Physiology 9: 2018.
23. Dimke H, Sparks M A, Thomson B R, Frische S, Coffman T M, and Quaggin S E. Tubulovascular cross-talk by vascular endothelial growth factor a maintains peritubular microvasculature in kidney. J Am Soc Nephrol 26: 1027-1038, 2015.
24. Eschbach J W, Jr., Funk D, Adamson J, Kuhn I, Scribner B H, and Finch C A. Erythropoiesis in patients with renal failure undergoing chronic dialysis. N Engl J Med 276: 653-658, 1967.
25. Fervenza F C, Hendry B M, and Ellory J C. Effects of dialysis and transplantation on red cell Na pump function in renal failure. Nephron 53: 121-128, 1989.
26. Fraenkel P G. Anemia of Inflammation: A Review. Med Clin North Am 101: 285-296, 2017.
27. Fudin R, Jaichenko J, Shostak A, Bennett M, and Gotloib L. Correction of Uremic Iron Deficiency Anemia in Hemodialyzed Patients: A Prospective Study. Nephron 79: 299-305, 1998.
28. Gwozdzinski K, Pieniazek A, and Gwozdzinski L. Reactive Oxygen Species and Their Involvement in Red Blood Cell Damage in Chronic Kidney Disease. Oxid Med Cell Longev 2021: 6639199, 2021.
29. Hentze M W, Muckenthaler M U, and Andrews N C. Balancing acts: molecular control of mammalian iron metabolism. Cell 117: 285-297, 2004.
30. Izumo H, Izumo S, DeLuise M, and Flier J S. Erythrocyte Na,K pump in uremia. Acute correction of a transport defect by hemodialysis. J Clin Invest 74: 581-588, 1984.
31. Kaji D, and Thomas K. Na+-K+ pump in chronic renal failure. Am J Physiol 252: F785-793, 1987.
32. Koc B, Erten V, Yilmaz M I, Sonmez A, and Kocar I H. The relationship between red blood cell Na/K-ATPase activities and diabetic complications in patients with type 2 diabetes mellitus. Endocrine 21: 273-278, 2003.
33. Kramer H J, Gospodinov D, and Kruck F. Functional and metabolic studies on red blood cell sodium transport in chronic uremia. Nephron 16: 344-358, 1976.
34. Kuhn V, Diederich L, Keller T CSt, Kramer C M, Luckstadt W, Panknin C, Suvorava T, Isakson B E, Kelm M, and Cortese-Krott M M. Red Blood Cell Function and Dysfunction: Redox Regulation, Nitric Oxide Metabolism, Anemia. Antioxid Redox Signal 26: 718-742, 2017.
35. Kuypers F A, Lewis R A, Hua M, Schott M A, Discher D, Ernst J D, and Lubin B H. Detection of altered membrane phospholipid asymmetry in subpopulations of human red blood cells using fluorescently labeled annexin V. Blood 87: 1179-1187, 1996.
36. Lang E, Bissinger R, Gulbins E, and Lang F. Ceramide in the regulation of eryptosis, the suicidal erythrocyte death. Apoptosis 20: 758-767, 2015.
37. Lang E, Qadri S M, and Lang F. Killing me softly—suicidal erythrocyte death. Int J Biochem Cell Biol 44:1236-1243, 2012.
38. Lang F, Abed M, Lang E, and Foller M. Oxidative stress and suicidal erythrocyte death. Antioxid Redox Signal 21: 138-153, 2014.
39. Lang F, and Qadri S M. Mechanisms and significance of eryptosis, the suicidal death of erythrocytes. Blood Purif 33: 125-130, 2012.
40. Lew V L, and Tiffert T. On the Mechanism of Human Red Blood Cell Longevity: Roles of Calcium, the Sodium Pump, PIEZO1, and Gardos Channels. Front Physiol 8: 977, 2017.
41. Li J H, Luo J F, Jiang Y, Ma Y J, Ji Y Q, Zhu G L, Zhou C, Chu H W, and Zhang H D. Red Blood Cell Lifespan Shortening in Patients with Early-Stage Chronic Kidney Disease. Kidney Blood Press Res 44: 1158-1165, 2019.
42. Li Z, Cai T, Tian J, Xie J X, Zhao X, Liu L, Shapiro J I, and Xie Z. NaKtide, a Na/K-ATPase-derived Peptide Src Inhibitor, Antagonizes Ouabain-activated Signal Transduction in Cultured Cells. Journal of Biological Chemistry 284: 21066-21076, 2009.
43. Li Z, and Xie Z. The Na/K-ATPase/Src complex and cardiotonic steroid-activated protein kinase cascades. Pflugers Arch 457: 635-644, 2009.
44. Li Z, Zhang Z, Xie J X, Li X, Tian J, Cai T, Cui H, Ding H, Shapiro J I, and Xie Z. Na/K-ATPase mimetic pNaKtide peptide inhibits the growth of human cancer cells. J Biol Chem 286: 32394-32403, 2011.
45. Liu J, Nie Y, Chaudhry M, Bai F, Chuang J, Sodhi K, and Shapiro J I. The Redox-Sensitive Na/K-ATPase Signaling in Uremic Cardiomyopathy. Int J Mol Sci 21: 2020.
46. Liu J, Tian J, Chaudhry M, Maxwell K, Yan Y, Wang X, Shah P T, Khawaja A A, Martin R, Robinette T J, El-Hamdani A, Dodrill M W, Sodhi K, Drummond C A, Haller S T, Kennedy D J, Abraham N G, Xie Z, and Shapiro J I. Attenuation of Na/K-ATPase Mediated Oxidant Amplification with pNaKtide Ameliorates Experimental Uremic Cardiomyopathy. Scientific Reports 6: 34592, 2016.
47. Lopez A, Cacoub P, Macdougall I C, and Peyrin-Biroulet L. Iron deficiency anaemia. Lancet 387: 907-916, 2016.
48. Martin D W, and Sachs J R. Cross-linking of the erythrocyte (Na+,K+)-ATPase. Chemical cross-linkers induce alpha-subunit-band 3 heterodimers and do not induce alpha-subunit homodimers. J Biol Chem 267: 23922-23929, 1992.
49. Maxwell K D, Chuang J, Chaudhry M, Nie Y, Bai F, Sodhi K, Liu J, and Shapiro J I. The potential role of Na—K-ATPase and its signaling in the development of anemia in chronic kidney disease. Am J Physiol Renal Physiol 320: F234-f242, 2021.
50. Modaresi A, Nafar M, and Sahraei Z. Oxidative stress in chronic kidney disease. Iran J Kidney Dis 9: 165-179, 2015.
51. Mohanty J, Nagababu E, and Rifkind J. Red blood cell oxidative stress impairs oxygen delivery and induces red blood cell aging. Frontiers in Physiology 5: 2014.
52. Pan X, Suzuki N, Hirano I, Yamazaki S, Minegishi N, and Yamamoto M. Isolation and characterization of renal erythropoietin-producing cells from genetically produced anemia mice. PloS One 6: e25839, 2011.
53. Pavlović-Kentera V. CGK, Djukanovid L. Pathophysiology of Erythropoiesis in Renal Diseases. Berlin, Heidelberg: Springer, 1989.
54. Podkowinska A, and Formanowicz D. Chronic Kidney Disease as Oxidative Stress- and Inflammatory-Mediated Cardiovascular Disease. Antioxidants (Basel) 9: 2020.
55. Pratt R D, Brickman C R, Cottrill C L, Shapiro J I, and Liu J. The Na/K-ATPase Signaling: From Specific Ligands to General Reactive Oxygen Species. Int J Mol Sci 19: 2600, 2018.
56. Pries A R, Secomb T W, and Gaehtgens P. Biophysical aspects of blood flow in the microvasculature. Cardiovasc Res 32: 654-667, 1996.
57. Qadri S M, Bissinger R, Solh Z, and Oldenborg P A. Eryptosis in health and disease: A paradigm shift towards understanding the (patho)physiological implications of programmed cell death of erythrocytes. Blood Rev 31: 349-361, 2017.
58. Radosinska J, and Vrbjar N. The role of red blood cell deformability and Na,K-ATPase function in selected risk factors of cardiovascular diseases in humans: focus on hypertension, diabetes mellitus and hypercholesterolemia. Physiol Res 65 Suppl 1: S43-54, 2016.
59. Ratliff B B, Abdulmahdi W, Pawar R, and Wolin M S. Oxidant Mechanisms in Renal Injury and Disease. Antioxid Redox Signal 25: 119-146, 2016.
60. Sodhi K, Maxwell K, Yan Y, Liu J, Chaudhry M A, Getty M, Xie Z, Abraham N G, and Shapiro J I. pNaKtide inhibits Na/K-ATPase reactive oxygen species amplification and attenuates adipogenesis. Science Advances 1: e1500781, 2015.
61. Sodhi K, Wang X, Chaudhry M A, Lakhani H V, Zehra M, Pratt R, Nawab A, Cottrill C L, Snoad B, Bai F, Denvir J, Liu J, Sanabria J R, Xie Z, Abraham N G, and Shapiro J I. Central Role for Adipocyte Na,K-ATPase Oxidant Amplification Loop in the Pathogenesis of Experimental Uremic Cardiomyopathy. J Am Soc Nephrol 31: 1746-1760, 2020.
62. Souma T, Suzuki N, and Yamamoto M. Renal erythropoietin-producing cells in health and disease. Frontiers in Physiology 6: 2015.
63. Thiagarajan P, and Tait J F. Binding of annexin V/placental anticoagulant protein I to platelets. Evidence for phosphatidylserine exposure in the procoagulant response of activated platelets. Journal of Biological Chemistry 265: 17420-17423, 1990.
64. Tozoni S S, Dias G F, Bohnen G, Grobe N, Pecoits-Filho R, Kotanko P, and Moreno-Amaral A N. Uremia and Hypoxia Independently Induce Eryptosis and Erythrocyte Redox Imbalance. Cell Physiol Biochem 53: 794-804, 2019.
65. van Nooten F E, Green J, Brown R, Finkelstein F O, and Wish J. Burden of illness for patients with non-dialysis chronic kidney disease and anemia in the United States: review of the literature. J Med Econ 13: 241-256, 2010.
66. Vos F E, Schollum J B, Coulter C V, Doyle T C, Duffull S B, and Walker R J. Red blood cell survival in long-term dialysis patients. Am J Kidney Dis 58: 591-598, 2011.
67. Wang X, Chaudhry M A, Nie Y, Xie Z, Shapiro J I, and Liu J. A Mouse ⅚th Nephrectomy Model That Induces Experimental Uremic Cardiomyopathy. J Vis Exp 2017.
68. Yamazaki S, Souma T, Hirano I, Pan X, Minegishi N, Suzuki N, and Yamamoto M. A mouse model of adult-onset anaemia due to erythropoietin deficiency. Nat Commun 4: 1950, 2013.
69. Yan Y, Shapiro A P, Haller S, Katragadda V, Liu L, Tian J, Basrur V, Malhotra D, Xie Z J, Abraham N G, Shapiro J I, and Liu J. Involvement of reactive oxygen species in a feed-forward mechanism of Na/K-ATPase-mediated signaling transduction. J Biol Chem 288: 34249-34258, 2013.
70. Yan Y, Shapiro A P, Mopidevi B R, Chaudhry M A, Maxwell K, Haller S T, Drummond C A, Kennedy D J, Tian J, Malhotra D, Xie Z J, Shapiro J I, and Liu J. Protein Carbonylation of an Amino Acid Residue of the Na/K-ATPase al Subunit Determines Na/K-ATPase Signaling and Sodium Transport in Renal Proximal Tubular Cells. J Am Heart Assoc 5: 2016.
71. Yoon Y S, Kim S Y, Koo W S, Choi E J, Chang Y S, Bang B K, and Moon H R. Erythrocyte sodium transport in dialyzed uremic patients. Korean J Intern Med 4: 9-17, 1989.

Additionally, throughout this document, various nucleic acid and/or amino acid sequences are mentioned. All such sequence are incorporated herein by reference, including the sequences set forth in the following list:

### SEQUENCE LISTING

### SEQ ID NO: 1

SATWLALSRIAGLCNRAVFQ

### SEQ ID NO:2

GRKKRRQRRRPPQ

### SEQ ID NO:3

RQIKIWFQNRRMKWK K

### SEQ ID NO:4

KKGKKGKK

### SEQ ID NO:5

GRKKRRQRRRPPQSATWLALSRIAGLCNRAVFQ

### SEQ ID NO:6

GRKKRRQRRRPPQACWIQNLSRSAGATVRLFLA

It will be understood that various details of the presently disclosed subject matter can be changed without departing from the scope of the subject matter disclosed herein. Furthermore, the description provided herein is for the purpose of illustration only, and not for the purpose of limitation.

What is claimed is:

1. A method for treating anemia, comprising
identifying a subject as having anemia characterized by a reduced number of red blood cells; and
administering a polypeptide antagonist of a Na/K ATPase/Src receptor complex to the subject, the polypeptide anatagonist having the sequence of SEQ ID NO: 1, or a functional fragment, and/or functional variant thereof, and the polypeptide antagonist further including a cell penetrating peptide.

2. The method of claim 1, wherein polypeptide antagonist comprises the sequence of SEQ ID NO: 5.

3. The method of claim 1, wherein the cell penetrating polypeptide has an amino acid sequence selected from the group consisting of SEQ ID NOS: 2-4.

4. The method of claim 1, wherein the administering step includes oral administration, transdermal administration, administration by inhalation, nasal administration, topical administration, intraaural administration, rectal administration, intravenous administration, intramuscular administration, subcutaneous administration, intravitreous administration, subconjunctival administration, intracameral administration, intraocular administration or combinations thereof.

5. The method of claim 1, wherein the polypeptide antagonist is administered in an amount sufficient to increase a hematocrit level in the subject.

6. The method of claim 1, wherein the polypeptide antagonist is administered in an amount sufficient to reduce an amount of reticulocytes in the subject.

7. The method of claim 1, wherein the polypeptide antagonist is administered in an amount sufficient to increase a half-life of a red blood cell in the subject.

8. The method of claim 1, wherein the polypeptide antagonist is administered in an amount sufficient to decrease an amount of eryptosis in the subject.

9. The method of claim 1, wherein the subject has kidney disease.

10. The method of claim 1, wherein the anemia is chronic kidney disease-induced anemia.

11. A method for increasing red blood cell half-life, comprising administering a polypeptide antagonist of a Na/K ATPase/Src receptor complex to a subject in need thereof, the polypeptide anatagonist having the sequence of SEQ ID NO: 1, or a functional fragment, and/or functional variant thereof, and the polypeptide antagonist further including a cell penetrating peptide.

12. The method of claim 11, wherein the polypeptide anatagonist comprises the sequence of SEQ ID NO: 5.

13. The method of claim 11, wherein the cell penetrating polypeptide has an amino acid sequence selected from the group consisting of SEQ ID NOS: 2-4.

14. The method of claim 11, wherein the administering step includes oral administration, transdermal administration, administration by inhalation, nasal administration, topical administration, intraaural administration, rectal administration, intravenous administration, intramuscular administration, subcutaneous administration, intravitreous administration, subconjunctival administration, intracameral administration, intraocular administration or combinations thereof.

15. The method of claim 11, wherein the polypeptide antagonist is administered in an amount sufficient to reduce an amount of reticulocytes in the subject.

16. The method of claim 11, wherein the polypeptide antagonist is administered in an amount sufficient to decrease an amount of eryptosis in the subject.

17. A method for increasing red blood cell half-life, comprising contacting a red blood cell with a polypeptide antagonist of a Na/K ATPase/Src receptor complex, the polypeptide anatagonist having the sequence of SEQ ID NO: 1, or a functional fragment, and/or functional variant thereof, and the polypeptide antagonist further including a cell penetrating peptide.

18. The method of claim 17, wherein the polypeptide anatagonist comprises the sequence of SEQ ID NO: 5.

19. The method of claim 17, wherein the cell penetrating polypeptide has an amino acid sequence selected from the group consisting of SEQ ID NOS: 2-4.


SEQUENCE LISTING (6 sequences)

SEQ ID NO: 1 - protein, 20 aa, Homo sapiens
SATWLALSRIAGLCNRAVFQ

SEQ ID NO: 2 - protein, 13 aa, Human immunodeficiency virus
GRKKRRQRRRPPQ

SEQ ID NO: 3 - protein, 16 aa, Drosophila melanogaster
RQIKIWFQNRRMKWKK

SEQ ID NO: 4 - protein, 8 aa, Homo sapiens
KKGKKGKK

SEQ ID NO: 5 - protein, 33 aa, Artificial Sequence
  TAT NaKtide Fusion Polypeptide
GRKKRRQRRRPPQSATWLALSRIAGLCNRAVFQ

SEQ ID NO: 6 - protein, 33 aa, Artificial Sequence
  scrambled peptide
GRKKRRQRRRPPQACWIQNLSRSAGATVRLFLA